(19) United States
(12) Patent Application Publication
(10) Pub. No.: US 2012/0244089 A1
(43) Pub. Date: Sep. 27, 2012

(54) USE OF THIAZOLE COMPOUND IN FLAVOR APPLICATIONS

(76) Inventors: David O. Agyemang, East Brunswick, NY (US); Kathryn A. Bardsley, Howell, NJ (US); Linda Psota-Kelty, Tinton Falls, NJ (US); Laurence Trinnaman, Montvale, NJ (US)

(21) Appl. No.: 13/490,893

(22) Filed: Jun. 7, 2012

Related U.S. Application Data
(63) Continuation-in-part of application No. 12/551,909, filed on Sep. 1, 2009.

(51) Int. Cl. A23L 1/221; A23L 2/56; A23L 1/168; A61Q 11/00; A23C 9/123; A61K 8/49; A61K 47/22; A23G 4/06; A23L 1/31

(52) U.S. Cl. 424/49; 426/535; 426/531; 426/3; 514/788

(57) ABSTRACT

The present invention has discovered that 2-ethyl-4-methyl-2,5-dihydro-thiazole possesses unexpected and advantageous flavor properties.

### STATUS OF RELATED APPLICATIONS

This application is a continuation-in-part of U.S. Ser. No. 12/551,909, filed Sep. 1, 2009, now pending, the content hereby incorporated by reference as if set forth in its entirety.

### FIELD OF THE INVENTION

The present invention relates to the novel use of a thiazole compound to enhance, improve or modify the flavor of orally consumable compositions, such as foodstuff, chewing gums, dental and oral hygiene products, and medicinal products.

### BACKGROUND OF THE INVENTION

There is an ongoing need in the flavor industry to identify new flavor compounds such as compounds that provide a fruity note. In searching for naturally-occurring flavor compounds, toasted sesame seed oil was analyzed and novel flavorful thiazole compounds were identified. These compounds may be used in a wide variety of flavor applications.

### SUMMARY OF THE INVENTION

The present invention has discovered that 2-ethyl-4-methyl-2,5-dihydro-thiazole represented by Formula I set forth below possesses unexpected advantageous use in enhancing, improving or modifying the flavor of orally consumable compositions, such as foodstuff, chewing gums, dental and oral hygiene products, and medicinal products.

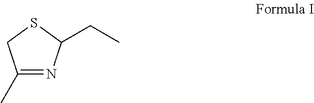

One embodiment of the invention relates to a flavor composition comprising 2-ethyl-4-methyl-2,5-dihydro-thiazole.

Another embodiment of the invention relates to a process of enhancing, improving or modifying the flavor of a material selected from the group consisting of foodstuff, a chewing gum, a dental or oral hygiene product, and a medicinal product comprising the step of incorporating an olfactory effective amount of 2-ethyl-4-methyl-2,5-dihydro-thiazole.

These and other embodiments of the present invention will be apparent by reading the following specification.

### DETAILED DESCRIPTION OF THE INVENTION

2-Ethyl-4-methyl-2,5-dihydro-thiazole has been surprisingly found to have unexpected properties of enhancing, improving and modifying flavors, which are demonstrated to be advantageous for augmenting or imparting taste enhancement or somatosensory effect in foodstuff, chewing gums, oral hygiene products, and medicinal products by providing flavor enhancement and a preferred overall flavor profile.

The terms “flavor composition” and “flavor formulation” are understood to mean the same and refer to a formulation that provides a flavor character to foodstuff, chewing gums, oral hygiene products, and medicinal products.

The term “improving” in the phrase “improving, enhancing or modifying the flavor of a material” is understood to mean raising the flavor of a material to a more desirable character. The term “enhancing” is understood to mean making the flavor of a material greater in effectiveness or providing the material with an improved flavor character. The term “modifying” is understood to mean providing the flavor of a material with a change in character.

As used herein, an olfactory effective amount is understood to mean the amount of the compound in a flavor composition that alters the characteristics of the composition, or enhances or modifies the flavor, taste, and aroma reaction contributed by another ingredient in the composition. The overall flavor, taste, and aroma effect of the composition will be the sum effect of all flavor ingredients. The olfactory effective amount will vary depending on many factors including other ingredients, their relative amounts, and the effect that is desired.

The usage level of the 2-ethyl-4-methyl-2,5-dihydro-thiazole varies depending on the product in which it is employed. Generally, the level of 2-ethyl-4-methyl-2,5-dihydro-thiazole employed in a product is greater than about 0.5 parts per billion by weight, preferably from about 4 parts per billion to about 50 parts per million by weight, more preferably from about 20 parts per billion to about 4 parts per million by weight.

As used herein, foodstuff includes both solid and liquid ingestible materials for man or animals, which materials usually do, but need not, have nutritional value. Thus, foodstuff includes food products, such as meats, gravies, soups, convenience foods, malt, alcoholic and other beverages, milk and dairy products such as yogurt, seafood, including fish, crustaceans, mollusks and the like, candies, vegetables, cereals, soft drinks such as sodas, snacks, dog and cat foods, other veterinary products and the like.

When 2-ethyl-4-methyl-2,5-dihydro-thiazole is used in an orally consumable composition, it can be combined with conventional flavoring ingredients or adjuvants, which are well known in the art. Requirements of such flavoring ingredients and adjuvants are that: (1) they be organoleptically compatible with the 2-ethyl-4-methyl-2,5-dihydro-thiazole whereby the flavor of the ultimate consumable composition to which the 2-ethyl-4-methyl-2,5-dihydro-thiazole is added is not detrimentally affected by the use of such flavoring ingredients and adjuvants; and (2) they be ingestible and thus nontoxic or otherwise non-deleterious. In addition, the orally consumable composition can broadly include other flavor materials, vehicles, stabilizers, thickeners, surface active agents, conditioners, and flavor intensifiers.

Such conventional flavoring ingredients include, for example, but are not limited to, saturated fatty acids, unsaturated fatty acids and amino acids; alcohols including primary and secondary alcohols, esters, carbonyl compounds including aldehydes and ketones; lactones; other cyclic organic materials including aryl compounds, alicyclic compounds, heterocyclics such as furans, pyridines, pyrazines and the like; sulfur-containing compounds including thiols, sulfides, disulfides and the like; proteins; lipids, carbohydrates; so-called flavor potentiators such as monosodium glutamate; magnesium glutamate, calcium glutamate, guanylates and inosinates; natural flavoring materials such as cocoa, vanilla and caramel; essential oils and extracts such as anise oil, clove oil and the like and artificial flavoring materials such as vanillin, ethyl vanillin and the like.

Such flavoring adjuvants include, for example, but are not limited to, anise oil; ethyl 2-methylbutyrate; vanillin; cis-3-heptenol; cis-3-hexenol; trans-2-heptenal; butyl valerate; 2,3-diethylpyrazine; methyl cyclopentenolone; benzaldehyde; valerian oil; 3,4-dimethoxyphenol; amyl acetate; amyl cinnamate; gamma-butyrolactone; furfural; trimethylpyrazine; phenylacetic acid; isovaleraldehyde; ethyl maltol; ethyl vanillin; ethyl valerate; ethyl butyrate; cocoa extract; coffee extract; peppermint oil; spearmint oil; clove oil; anethole; cardamom oil; wintergreen oil; cinnamic aldehyde; ethyl 2-methylvalerate; gamma-hexenyl lactone; 2,4-heptadienal; methyl thiazole alcohol (4-methyl-5-.beta.-hydroxyethyl thiazole); 2-methylbutanethiol; 4-mercaptobutan-2-one; 3-mercaptopentan-2-one; 1-mercapto-2-propene; benzaldehyde; furfural; furfuryl alcohol; 2-mercaptopropionic acid; alkyl pyrazine; methylpyrazine; 2-ethyl-3-methylpyrazine; tetramethylpyrazine; polysulfides; dipropyl disulfide; benzyl methyl disulfide; alkyl thiophene; 2,3-dimethylthiophene; 5-methylfurfural; 2-acetylfuran; 2,4-decadienal; guiacol; phenylacetaldehyde; beta-decalactone; d-limonene; acetoin; amyl acetate; maltol; ethyl butyrate; levulinic acid; piperonal; ethyl acetate; octanal; valeraldehyde; hexanal; diacetyl; monosodium gulatamate; monopotassium glutamate; sulfur-containing amino acids, e.g., cysteine; hydrolyzed vegetable protein; 2-methylfuran-3-thiol; 2-methyldihydrofuran-3-thiol; 2,5-dimethylfuran-3-thiol; hydrolyzed fish protein; tetramethylpyrazine; propylpropenyl disulfide; propylpropenyl trisulfide; diallyldisulfide; diallyltrisulfide; dipropenyl disulfide; dipropenyl trisulfide; 4-methyl-2-[(methylthio)-ethyl]-1,3-dithiolane; 4,5-dimethyl-2-(methylthiomethyl)-1,3-dithiolane; and 4-methyl-2-(methylthiomethyl)-1,3-dithiolane. These and other flavor ingredients are provided in U.S. Pat. Nos. 6,110,520 and 6,333,180 hereby incorporated by reference.

Such vehicles can be edible or otherwise suitable, which include, for example, but are not limited to, ethyl alcohol, propylene glycol, water and the like, as described supra. 2-Ethyl-4-methyl-2,5-dihydro-thiazole can also be incorporated into a carrier using conventional means such as spray-drying, drum-drying and the like. Such carriers include, for example, but are not limited to, gum arabic, carrageenan, xanthan gum, guar gum and the like. Such carriers can also include materials for coacervating 2-ethyl-4-methyl-2,5-dihydro-thiazole to provide encapsulated products, as set forth supra. When the carrier is an emulsion, the flavor composition can also contain emulsifiers such as mono- and diglycerides or fatty acids and the like. With these carriers or vehicles, the desired physical form of the compositions can be prepared.

The following are provided as specific embodiments of the present invention. Other modifications of this invention will be readily apparent to those skilled in the art. Such modifications are understood to be within the scope of this invention. As used herein all percentages are weight percent unless otherwise noted, g is understood to stand for gram, mol is understood to stand for mole, L is understood to be liter, mL is understood to stand for milliliter, ppb is understood to stand for parts per billion, and ppm is understood to stand for parts per million.

### Example I

### Preparation of 1-Mercapto-2-propanone

Chloroacetone (CH₃COCH₂Cl) (250 g, 2.70 mol) was fed into a solution of stirring sodium hydrosulfide hydrate (NaSH) (303 g, 5.40 mol), H₂O (300 mL), and ethanol (CH₃CH₂OH) (300 mL) at 10° C. for an hour. After the feeding was complete, the reaction mixture was stirred for additional 15 minutes. The reaction mixture was quenched with H₂O (250 mL) and then extracted with diethyl ether ((C₂H₅)₂O) (250 mL). The organic layer was washed with water followed by brine to obtain the crude product 1-mercapto-2-porpanone.

### Example II

### Preparation of 2-Ethyl-4-methyl-2,5-dihydro-thiazole (Formula I)

Ammonium hydroxide (NH₄OH) (30%, 630.9 g, 5.40 mol) was slowly added to a solution of propionaldehyde (CH₃CH₂CHO) (188 g, 3.24 mol) in tetrahydrofuran (THF) (9.5 L) while the temperature was maintained at 20° C. Once the addition completed, the reaction mixture was stirred for 15 minutes. Crude 1-mercapto-2-propanone (prepared as above in EXAMPLE I) was fed dropwise into the reaction mixture. Once the feeding was complete, the reaction mixture was further stirred for 15 minutes. The aqueous was separated and extracted with diethyl ether. The organic layers were then combined and washed with brine. The crude solution was concentrated in vacuum and purified by fractional distillation under a reduced pressure to afford 2-ethyl-4-methyl-2,5-dihydro-thiazole (129 g).

¹H NMR (CDCl₃, 500 MHz): 5.55-5.58 ppm (m, 1H), 3.84 ppm (m, 2H), 2.12 ppm (s, 3H), 1.95-2.03 ppm (m, 1H), 1.71-1.77 ppm (m, 1H), 1.00 ppm (t, 3H, J=7.35 Hz)

2-Ethyl-4-methyl-2,5-dihydro-thiazole in water (1 ppb) was described as having tropical fruit, catty, peach, and berry flavor notes.

### Example III

### Preparation of 4-Ethyl-2-methyl-3-thiazole (Formula II)

4-Ethyl-2-methyl-2,5-dihydro-thiazole represented by Formula II set forth below was similarly prepared as 2-ethyl-4-methyl-2,5-dihydro-thiazole in EXAMPLE I and EXAMPLE II.

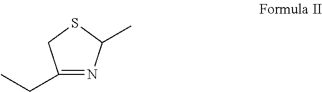

¹H NMR (CDCl₃, 500 MHz): 5.55-5.58 ppm (m, 1H), 3.86-4.02 ppm (m, 2H), 2.34 ppm (q, 2H, J=7.45 Hz), 1.46 ppm (d, 3H, J=6.45 Hz), 1.09 ppm (t, 3H, J=7.45 Hz)

### Example IV

### Preparation of 4-Methyl-2-propyl-2,5-dihydro-thiazole (Formula III)

4-Methyl-2-propyl-2,5-dihydro-thiazole represented by Formula III set forth below was similarly prepared as 2-ethyl-4-methyl-2,5-dihydro-thiazole in EXAMPLE I and EXAMPLE II.

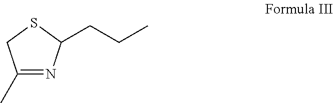

¹H NMR (CDCl₃, 500 MHz): 5.54-5.64 ppm (m, 1H), 3.78-3.90 ppm (m, 2H), 2.13 ppm (s, 3H), 1.94-2.02 ppm (m, 1H), 1.64-1.71 ppm (m, 1H), 1.39-1.53 ppm (m, 2H), 0.96 ppm (t, 3H, J=7.35 Hz)

### Example V

The flavor properties of 2-ethyl-4-methyl-2,5-dihydro-thiazole were evaluated in an oatmeal sample. Commercially available plain instant oatmeal was used whose ingredients included whole grain rolled oats, calcium carbonate, salt, guar gum, caramel color, reduced iron, niacinamide, vitamin A, palmitate, pyridoxine, hydrochloride, riboflavin, thiamin mononitrate, and folic acid.

2-Ethyl-4-methyl-2,5-dihydro-thiazole was described as having added faint sulfurous note and synergized with grain note at 1 ppb, and sulfurous and peach notes at 5 ppb.

### Example VI

The flavor properties of 2-ethyl-4-methyl-2,5-dihydro-thiazole were evaluated in a soft drink sample. An orange soda was used whose ingredients included carbonated water, high fructose corn syrup, citric acid, potassium benzoate, gum acacia, natural flavor, ester gum, yellow 6, brominated vegetable oil, and red 40.

2-Ethyl-4-methyl-2,5-dihydro-thiazole (5 ppb) was described as having provided the feel of a tangerine soda and more natural taste and having cut the acidity.

### Example VII

The flavor properties of 2-ethyl-4-methyl-2,5-dihydro-thiazole were evaluated in a beef broth sample. The beef broth sample included beef broth and less than 1% of the following: salt, MSG, hydrolyzed corn protein, hydrolyzed soy protein, hydrolyzed wheat protein, dextrose, onion powder, autolyzed yeast extract, caramel color, partially hydrogenated soybean oil, natural flavor, thiamine hydrochloride, disodium inosinate, and disodium guanylate.

2-Ethyl-4-methyl-2,5-dihydro-thiazole (4 ppb) was described as having boosted the roast note and imparted additional tropical note.

### Example VIII

The flavor properties of 2-ethyl-4-methyl-2,5-dihydro-thiazole were evaluated in a dairy product sample. A mixed berry yogurt sample was used whose ingredients included cultured pasteurized grade A nonfat milk, high fructose corn syrup, modified corn starch, nonfat milk, kosher gelatin, corn starch, tricalcium phosphate, natural flavor, potassium sorbate added to maintain freshness, sucralose (splenda brand,) acesulfame potassium, colored with annatto extract, vitamin A acetate, and vitamin D3.

2-Ethyl-4-methyl-2,5-dihydro-thiazole (5 ppb) was described as having boosted the fruity note and imparted a peach note.

### Example IX

The flavor properties of 2-ethyl-4-methyl-2,5-dihydro-thiazole (Formula I), 4-ethyl-2-methyl-3-thiazole (Formula II), and 4-methyl-2-propyl-2,5-dihydro-thiazole (Formula III) in water were evaluated by a trained sensory panel using the 9-point hedonic scale. The hedonic values were reported in the following:

| | | | Flavor |
|---|---|---|---|
| Compound | Concentration | Flavor Profile | Intensity |
| Formula I | 100 ppb | Tropical, fruit, peach, | 7.11 |
| | | slightly catty, and meaty. | |
| | | Very strong. | |
| Formula II | 1 ppm | Nutty and cabbage | 1 |
| Formula III | 350 ppb | Sulfury, nutty, and skunk. | 3.33 |
| | | Strong. | |

2-Ethyl-4-methyl-2,5-dihydro-thiazole (Formula I) exhibited unexpected strong and long-lasting fruity character. Even at a lower concentration, Formula I was shown to be superior to all other structurally closely related analogs.

What is claimed is:

1. A flavor composition comprising 2-ethyl-4-methyl-2,5-dihydro-thiazole, wherein 2-ethyl-4-methyl-2,5-dihydro-thiazole is provided at a level of greater than about 0.5 part per billion by weight.

2. The flavor composition of claim 1 further incorporated into a material selected from the group consisting of a foodstuff, a chewing gum, a dental or oral hygiene product, and a medicinal product.

3. The flavor composition of claim 2, wherein the material is the foodstuff.

4. The flavor composition of claim 3, wherein the foodstuff is an oatmeal.

5. The flavor composition of claim 3, wherein the foodstuff is a soft drink.

6. The flavor composition of claim 3, wherein the foodstuff is a meat.

7. The flavor composition of claim 3, wherein the foodstuff is a yogurt.

8. The flavor composition of claim 1, wherein 2-ethyl-4-methyl-2,5-dihydro-thiazole is provided at a level of from about 4 parts per billion to about 50 parts per million by weight.

9. The flavor composition of claim 1, wherein 2-ethyl-4-methyl-2,5-dihydro-thiazole is provided at a level of from about 20 parts per billion to about 4 parts per million by weight.

10. A process of improving, enhancing or modifying the flavor of a material selected from the group consisting of a foodstuff, a chewing gum, a dental or oral hygiene product, and a medicinal product comprising the step of incorporating an effective amount of 2-ethyl-4-methyl-2,5-dihydro-thiazole, wherein the effective amount is greater than about 0.5 parts per billion by weight.

11. The process of claim 10, wherein the material is the foodstuff.

12. The process of claim 11, wherein the foodstuff is an oatmeal.

13. The process of claim 11, wherein the foodstuff is a soft drink.

14. The process of claim 11, wherein the foodstuff is a meat.

15. The process of claim 11, wherein the foodstuff is a yogurt.

16. The process of claim 10, wherein the effective amount is from about 4 parts per billion to about 50 parts per million by weight.

17. The process of claim 10, wherein effective amount is from about 20 parts per billion to about 4 parts per million by weight.
